(19) United States
(12) Patent Application Publication
(10) Pub. No.: US 2010/0305543 A1
(43) Pub. Date: Dec. 2, 2010

(54) NONWOVEN FABRIC AND METHOD FOR THE PRODUCTION THEREOF

(76) Inventor: Frantisek Klaska, Slavkov U Brna (CZ)

Correspondence Address: DLA PIPER LLP (US);ATTN: PATENT GROUP, P.O. Box 2758, Reston, VA, 20195, US

(21) Appl. No.: 12/673,681

(22) PCT Filed: Aug. 18, 2008
(86) PCT No.: PCT/CZ08/00093

§ 371 Date: Mar. 24, 2010

(30) Foreign Application Priority Data
Aug. 16, 2007 (CZ) PV 2007-554

(51) Int. Cl. A61F 13/62; D04H 3/14; B32B 3/06; B29C 47/92

(52) U.S. Cl. 604/391; 428/198; 428/171; 264/175

(57) ABSTRACT

Nonwoven fabrics of a basis weight of 10 to 30 g/m² prepared from endless filaments of 0.5 to 3.5 den of fineness by spun-melt technology primarily for producing hygienic products. It consists of endless polyolefin filaments of a volume mass in the range from 40 to 80 g/m³, its basis weight is in the range from 10 to 30 g/m² and its thickness is in the range from 0.15 to 0.80 mm, being thermally bonded on a part of its surface in the range from 10 to 16% of its total surface area. The method for producing the nonwoven fabrics where a basic system of endless filaments of 0.5 to 3.5 den of fineness is created by spun-melt technology, which is stochastically deposited on a moving belt, of a basis weight of 10 to 30 g/m² and then it passes through a calendering system consisting of a smooth roller and an embossed roller.

### FIELD OF THE INVENTION

The present invention relates to nonwoven fabric of a basis weight of 10 to 30 g/m² prepared from endless filaments of 0.5 to 3.5 den of fineness by spun-melt technology primarily for producing hygienic products and method of production thereof.

### BACKGROUND OF THE INVENTION

In the manufacture of hygienic absorbent products as diapers for children, sanitary napkins and incontinence pads for patients, mostly nonwoven fabrics are used for the construction of these products prepared by spun-melt technology from endless polyolefin filaments, i.e. spunbonding and producing sheeting layers of the product. On the one hand the top-sheet, on the other the back-sheet. The basis weight of these nonwoven fabrics is usually in the range from 10 to 30 g/m2 and their thickness in the range from 0.15 to 0.25 mm. The achieved ratio of basis weight and thickness, that is the volume mass, indicates the bulk of the product and in this case it is in the range from 40 to 200 kg/m³. These processes are described for example in patents U.S. Pat. No. 5,814,349 a U.S. Pat. No. 5,814,249 (Reifenhäuser).

The nonwoven fabrics currently used for hygienic products are thermally reinforced by raster calender, making use of a raster pattern with a bonding area of 17 to 25% of the total area of the raster calender.

An often used raster for this type of nonwoven fabrics is the so-called “pyramid” pattern, available from the manufacturer of the embossed surfaces the company Ungericht under the trade designation U 2888. This known and current method of thermal reinforcement of nonwoven fabrics makes it possible to produce sufficiently durable nonwoven fabric, nevertheless, its drawback consists in the fact that such nonwoven fabrics are not sufficiently bulky, sufficiently elastic, sufficiently soft and sufficiently flexible, which manifests itself by general stiffness of the final product as diapers, hygienic napkins and incontinence pads.

### SUBJECT MATTER OF THE INVENTION

The subject matter of the nonwoven fabric according to the present invention consists in the fact that it is composed of endless polyolefin filaments of a volume mass in the range from 40 to 80 g/m³, basis weight in the range from 10 to 30 g/m² and thickness in the range from 0.15 to 0.80 mm, thermally bonded on a part of its surface in the range from 10 to 16% of its total surface area.

The polyolefin filaments are preferably monomeric or bicomponent filaments.

To achieve the required bulk, it is essential that it is thermobonded on a part of its surface, which amounts to 10 to 16% of its total surface area, the bonding area being usually equal to 14% and the whole nonwoven fabric is treated with hydrophilic lubrication, for example with a lubricating agent.

It is an advantage that the thermal bonding is accomplished in the form of longitudinal spots distributed in the shape of a raster, where each adjacent line is offset with respect to both adjacent lines by a half of the longitudinal pitch of the raster. The pitch of the individual bonding spots in the longitudinal direction is equal to 2 mm and in the transversal direction of 5.58 mm, the width of the bonding spot is 0.4 mm and its length is equal to 3.99 mm, the number of points per square cm is 9 points, the bonding spot v is 1.562 mm², the raster depth is 0.7 mm.

The method for producing the nonwoven fabric according to the present invention consists in the fact that a basic system of endless filaments of 0.5 to 3.5 den of fineness is created by spun-melt technology, which is stochastically deposited on a moving belt, of a basis weight of 10 to g/m² and then is passes through a calendering system consisting of a smooth roller and an embossed roller.

The temperature of the embossed roller is preferably in the range from 110 to 190° C. and the pressing force of the embossed roller on the smooth roller is in the range from 50 to 100 N/mm and the endless filaments are produced by monopolymeric polyolefin endless filaments.

The advantage consists in the fact that nonwoven fabric is used as a covering back-sheet in the final hygienic product and, at the same time, as a loop counter-fastener in the fastening system “hook-loop”.

The invention solves to a considerable extent the above-mentioned disadvantages of the prior art, namely the high stiffness, insufficient softness and compliance of the nonwoven fabric. The method according to the present invention provides a very bulky, soft, nonwoven fabric of low stiffness, loose structure, with a very good hand and good flexibility, and with high stretching capacity even in the transversal direction. A pattern with smaller bonding area and with optimal distribution of bonding points on the calender embossed roller has been applied.

### BRIEF DESCRIPTION OF FIGURES IN DRAWINGS

FIG. 1 illustrates the pattern of an embossed roller with dimensions specified in mm, FIG. 2 illustrates secion A-A indicated in FIG. 1 with dimensions specified in mm and FIG. 3 illustrates the appearance of the nonwoven fabric according to the present invention.

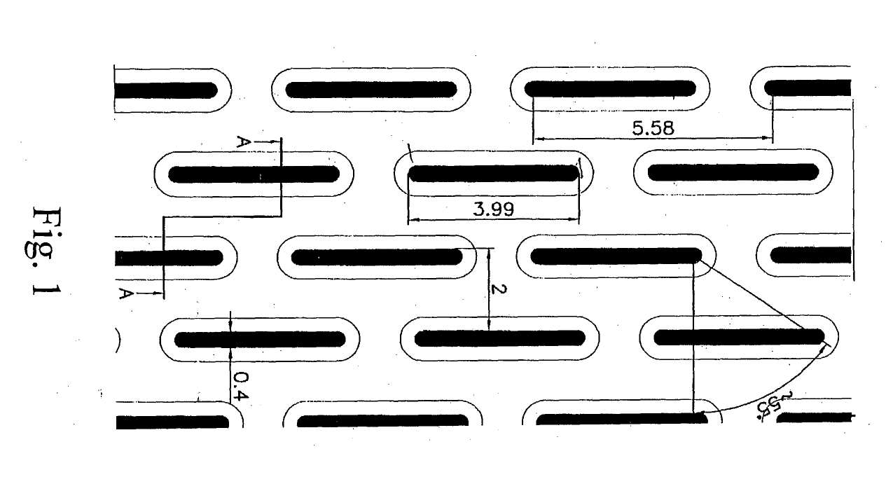

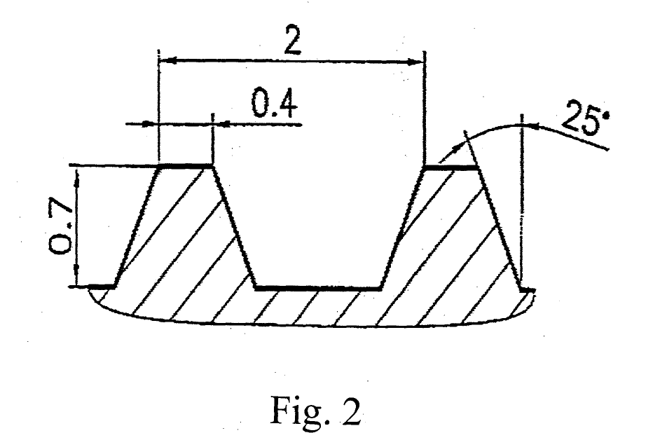

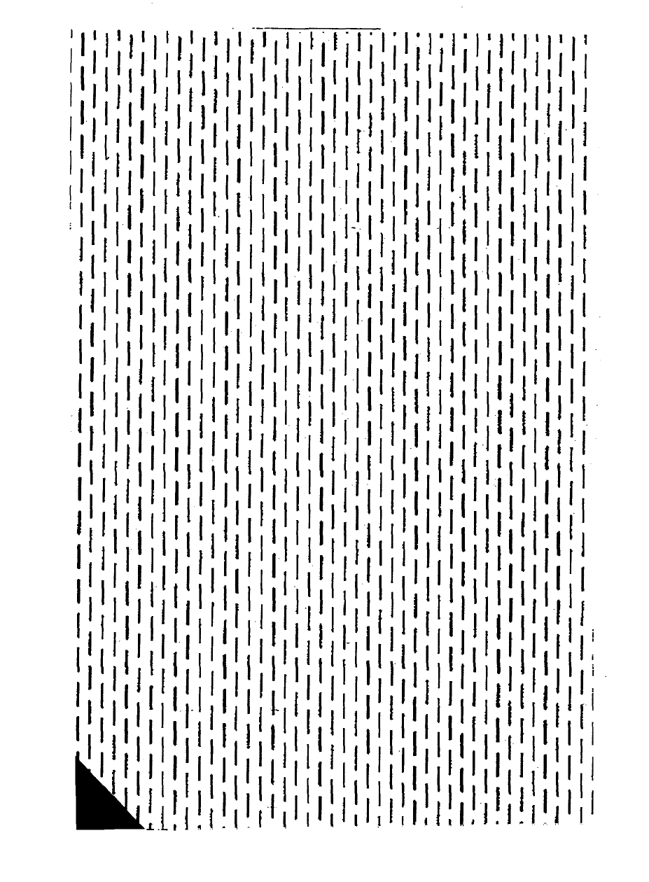

### EXAMPLES OF INVENTION EMBODIMENTS

The spun-melt nonwoven fabric according to the present invention is prepared from endless filaments, for example monopolymeric polyolefin filaments of required basis weight stochastically deposited on a moving belt. The filament fineness is within a range of 0.5 to 3.5 den, most frequently higher than 1.0 den, and the basis weight is within the range of 10 to 30 g/m².

In the known process of nonwoven fabric production of spun-melt type, the polymer granules are hot melted to obtain melt and consequently extruded through melt-spinning nozzles to produce a number of fine filaments. The air nozzles provide the elongation of filaments, which are deposited on a moving belt to form a stochastically arranged flat filament configuration.

The produced flat filament configuration passes through the bonding device constituted by bonding calender rollers heated up to a temperature of 110 to 190° C. adjusted to a pressure of 50 to 100 N/mm. One of the pair of bonding rollers is provided with a bonding raster produced by a series of elevated bonding areas—bonding points.

The pattern of the embossed calender roller is constituted by a series of parallel longitudinally arranged bonding points whose bonding area is in range of 10 to 16% of the surface area of the calender roller, the preferred bonding area being 14% of the surface area of the calender roller.

We have used the pattern designated by the manufacturer of raster surfaces Ungricht by trade designation U 3343B. As illustrated in FIG. 2, the most important dimensions of this pattern are as follows:
bonding area 14%
number of points per cm² is 9 points
bonding area in /mm²/ is 1.562 mm²
depth of engraving is 0.7 mm

Thermal bonding of the nonwoven fabric is in the form of longitudinal spots distributed on the latter in the shape of a raster, where each adjacent line is offset with respect to both adjacent lines by a half of the raster longitudinal pitch.

Volume mass is the function of ratio of basis weight and thickness—when the basis weight is maintained at a constant value and the thickness is less, the nonwoven fabric is bulkier and its volume mass is less and vice versa.

In the case the basis weight is 14 g/m², the volume mass is lower by 17%, in the case the basis weight is 17 g/m², the volume mass is lower by 27%, in the case the basis weight is 20 g/m², the volume mass is lower by 32% than in nonwoven fabrics of comparable basis weight reinforced by a current pattern U 2888.

According to another embodiment, the nonwoven fabric is manufactured from endless filaments produced from two polymers, the polymers being polyolefins. The cross section of the filament may be in the form of core/sheath or side/side, the abundance of the individual polymers being in the range of 10 to 90%, for example polymer A 90% and polymer B 10% and vice versa.

By creating a loose structure of the nonwoven fabric, the invention enables a simplified embodiment of fasteners of hygienic products of the “hook-loop” type. In the standard hook-type fasteners, one part of the fastener comprises hooks and the other part, i.e. the counter-fastener, consists of a strip with loosened fibers serving as loop-like fastening elements into which the hooks engage. The application of nonwoven fabrics according to the present invention enables fastening without the use of a counter-fastener, which is substituted by the voluminous nonwoven fabric according to the present invention.

Specific examples of nonwoven fabric embodiments according to the present invention:

### Example 1

The nonwoven fabric is made from polypropylene filaments with a fineness of 1.5 den. Its basis weight is 14 g/m² and its thickness is 0.23 mm, its volume mass thus being 60.86 kg/m³.

To reinforce it, a raster calender was used provided with a roller with pattern U 3343B described above and the applied technological characteristics were as follows:
Calender rollers temperature (smooth roller/embossed roller): 147/155° C.
Pressure of both rollers: 70 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 31.7 |
| Transversal tensile strength | EN 29073 T3 | N/50 mm | 14.1 |
| Longitudinal elongation at break | EN 29073 T3 | % | 81.1 |
| Transversal elongation at break | EN 29073 T3 | % | 77.1 |
| Bending stiffness (Handle-O-Meter) | IST 90.3.2001 | mN | 139.8 |

where EN designates a European Standard
IST designates an international standard according to the American Association of the Nonwoven Fabrics Industry INDA.

### Example 2

The nonwoven fabric is made from polypropylene filaments of a fineness of 1.5 den. Its basis weight is 14 g/m² and its thickness is 0.23 mm, its volume mass thus being 60.86 kg/m³.

The nonwoven fabric is treated with hydrophilic lubrication, for example with lubricating agent Silastol PHP 90 from the company Schill&Seilacher, in the amount of 0.40% by weight of the material.

To reinforce it, a raster calender was used provided with a roller with U 3343B pattern and the applied technological characteristics were as follows:
Calender rollers temperature (smooth roller/embossed roller): 147/155° C.
Pressure of both rollers: 70 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 31.7 |
| Transversal tensile strength | EN 29073 T3 | N/50 mm | 14.1 |
| Longitudinal elongation at break | EN 29073 T3 | % | 81.1 |
| Transversal elongation at break | EN 29073 T3 | % | 77.1 |
| Bending stiffness | IST 90.3.2001 | mN | 139.8 |
| Liquid strike-through time | EDANA 150.5-02 | s | 2.04 |
| Run-off | WSP 80.9 (05) | g | 0.00 |

where EDANA is the European Disposables And Nonwovens Association
The standards of both Associations are gradually unified and designated as:
WSP, which means the international standard of the World Strategic Partners

### Example 3

The nonwoven fabric is made from polypropylene filaments of a fineness of 1.5 den. Its basis weight is 17 g/m² and its thickness is 0.28 mm, its volume mass thus being 60.71 kg/m³.

To reinforce it, a raster calender was used provided with a roller with U 3343B pattern and the applied technological characteristics were as follows:
Calender rollers temperature (smooth roller/embossed roller): 142/147° C.
Pressure of both rollers: 73 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 34.7 |
| Transversal tensile strength | EN 29073 T3 | N/50 mm | 18.0 |
| Longitudinal elongation at break | EN 29073 T3 | % | 82.3 |
| Transversal elongation at break | EN 29073 T3 | % | 83.8 |
| Bending stiffness | IST 90.3.2001 | mN | 202.2 |

### Example 4

The nonwoven fabric is made from polypropylene filaments of a fineness of 1.5 den. Its basis weight is 17 g/m² and its thickness is 0.28 mm, its volume mass thus being 60.71 kg/m³.

The nonwoven fabric is treated with hydrophilic lubricating agent Silastol PHP 90 from the company Schill&Seilacher, in the amount of 0.40% by weight of the material.

To reinforce it, a raster calender was used provided with a roller with U 3343B pattern and the applied technological characteristics were as follows:
Calender rollers temperature (smooth roller/embossed roller): 142/147° C.
Pressure of both rollers: 73 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 34.7 |
| Transversal tensile strength | EN 29073 T3 | N/50 mm | 18.0 |
| Longitudinal elongation at break | EN 29073 T3 | % | 82.3 |
| Transversal elongation at break | EN 29073 T3 | % | 83.8 |
| Bending stiffness | IST 90.3.2001 | mN | 202.2 |
| Liquid strike-through time | EDANA 150.5-02 | s | 2.24 |
| Run-off | WSP 80.9 (05) | g | 0.00 |

### Example 5

The nonwoven fabric is made from polypropylene filaments of a fineness of 1.5 den. Its basis weight is 20 g/m² and its thickness is 0.31 mm, its volume mass thus being 64.51 kg/m³.

To reinforce it, a raster calender was used provided with a roller with U 3343B pattern and the applied technological characteristics were as follows:
Calender rollers temperature (smooth roller/embossed roller): 154/163° C.
Pressure of both rollers: 75 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 42.3 |
| Transversal tensile strength | EN 29073 T3 | N/50 mm | 23.2 |
| Longitudinal elongation at break | EN 29073 T3 | % | 82.3 |
| Transversal elongation at break | EN 29073 T3 | % | 86.3 |
| Bending stiffness | IST 90.3.2001 | mN | 269.0 |

### Example 6

The nonwoven fabric is made from polypropylene filaments of a fineness of 1.5 den. Its basis weight is 20 g/m² and its thickness is 0.31 mm, its volume mass thus being 64.51 kg/m³.

The nonwoven fabric is treated with hydrophilic lubrication, for example with lubricating agent Silastol PHP 90 from the company Schill&Seilacher, in the amount of 0.40% by weight of the material.

To reinforce it, a raster calender was used provided with a roller with U 3343B pattern and the applied technological characteristics were as follows:
Calender rollers temperature (smooth roller/embossed roller): 154/163° C.
Pressure of both rollers: 75 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 42.3 |
| Transversal tensile strength | EN 29073 T3 | N/50 mm | 23.2 |
| Longitudinal elongation at break | EN 29073 T3 | % | 82.3 |
| Transversal elongation at break | EN 29073 T3 | % | 86.3 |
| Bending stiffness | IST 90.3.2001 | mN | 269.0 |
| Liquid strike-through time | EDANA 150.5-02 | s | 2.49 |
| Run-off | WSP 80.9 (05) | g | 0.00 |

### Example 7

The nonwoven fabric is made from polypropylene and polyethylene filaments in the form of core/sheath in the ratio of 70% of polypropylene and 30% of polyethylene of a fineness of 1.5 den. Its basis weight is 12 g/m² and its thickness is 0.17 mm, its volume mass thus being 70.58 kg/m³.

To reinforce it, a raster calender was used provided with a roller with U 3343B pattern and the applied technological characteristics were as follows:
Calender rollers temperature (smooth roller/embossed roller): 132/136° C.
Pressure of both rollers: 62 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 20.0 |
| Transversal tensile strength | EN 29073 T3 | N/50 mm | 6.0 |
| Longitudinal elongation at break | EN 29073 T3 | % | 50.5 |
| Transversal elongation at break | EN 29073 T3 | % | 80.7 |
| Bending stiffness | IST 90.3.2001 | mN | 101.6 |

### Example 8

The nonwoven fabric is made from polypropylene and polyethylene filaments in the form of core/sheath in the ratio of 70% of polypropylene and 30% of polyethylene of a fineness of 1.5 den Its basis weight is 12 g/m² and its thickness is 0.17 mm, its volume mass thus being 70.58 kg/m³.

The nonwoven fabric is treated with hydrophilic lubrication, for example with lubricating agent Silastol PHP 90 from the company Schill&Seilacher, in the amount of 0.40% by weight of the material.

To reinforce it, a raster calender was used provided with a roller with U 3343B pattern and the applied technological characteristics were as follows:
Calender rollers temperature (smooth roller/embossed roller): 132/136° C.
Pressure of both rollers: 62 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 20.0 |
| Transversal tensile strength | EN 29073 T3 | N/50 mm | 6.0 |
| Longitudinal elongation at break | EN 29073 T3 | % | 50.5 |
| Transversal elongation at break | EN 29073 T3 | % | 80.7 |
| Bending stiffness | IST 90.3.2001 | mN | 101.6 |
| Liquid strike-through time | EDANA 150.5-02 | s | 2.09 |
| Run-off | WSP 80.9 (05) | g | 0.00 |

### Example 9

The nonwoven fabric is made from polypropylene and polyethylene filaments in the form of core/sheath in the ratio of 70% of polypropylene and 30% of polyethylene of a fineness of 1.5 den. Its basis weight is 14 g/m² and its thickness is 0.18 mm, its volume mass thus being 77.77 kg/m³.

To reinforce it, a raster calender was used provided with a roller with U 3343B pattern and the applied technological characteristics were as follows:
Calender rollers temperature (smooth roller/embossed roller): 134/138° C.
Pressure of both rollers: 63 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 26.8 |
| Transversal tensile strength | EN 29073 T3 | N/50 mm | 1.4 |
| Longitudinal elongation at break | EN 29073 T3 | % | 25.2 |
| Transversal elongation at break | EN 29073 T3 | % | 30.2 |
| Bending stiffness | IST 90.3.2001 | mN | 146.6 |

### Example 10

The nonwoven fabric is made from polypropylene and polyethylene filaments in the form of core/sheath in the ratio of 70% of polypropylene and 30% of polyethylene of a fineness of 1.5 den. Its basis weight is 14 g/m² and its thickness is 0.18 mm, its volume mass thus being 77.77 kg/m³.

The nonwoven fabric is treated with hydrophilic lubricating agent Silastol PHP 90 from the company Schill&Seilacher, in the amount of 0.40% by weight of the material.

To reinforce it, a raster calender was used provided with a roller with U 3343B pattern and the applied technological characteristics were as follows:
Calender rollers temperature (smooth roller/embossed roller): 134/138° C.
Pressure of both rollers: 63 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 26.8 |
| Transversal tensile strength | EN 29073 T3 | N/50 mm | 1.4 |
| Longitudinal elongation at break | EN 29073 T3 | % | 25.2 |
| Transversal elongation at break | EN 29073 T3 | % | 30.2 |
| Bending stiffness | IST 90.3.2001 | mN | 146.6 |
| Liquid strike-through time | EDANA 150.5-02 | s | 1.98 |
| Run-off | WSP 80.9 (05) | g | 0.00 |

### Example 11

The nonwoven fabric is made from polypropylene and polyethylene filaments in the form of core/sheath in the ratio of 70% of polypropylene and 30% of polyethylene of a fineness of 1.5 den. Its basis weight is 17 g/m² and its thickness is 0.18 mm, its volume mass thus being 9 kg/m³.

To reinforce it, a raster calender was used provided with a roller with U 3343B pattern and the applied technological characteristics were as follows:
Calender rollers temperature (smooth roller/embossed roller): 146/148° C.
Pressure of both rollers: 73 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 26.2 |
| Transversal tensile strength | EN 29073 T3 | N/50 mm | 10.6 |
| Longitudinal elongation at break | EN 29073 T3 | % | 65.5 |
| Transversal elongation at break | EN 29073 T3 | % | 96.5 |
| Bending stiffness | IST 90.3.2001 | mN | 153.4 |

### Example 12

The nonwoven fabric is made from polypropylene and polyethylene filaments in the form of core/sheath in the ratio of 70% of polypropylene and 30% of polyethylene of a fineness of 1.5 den. Its basis weight is 17 g/m² and its thickness is 0.18 mm, its volume mass thus being 9 kg/m³.

The nonwoven fabric is treated with hydrophilic lubricating agent Silastol PHP 90 from the company Schill&Seilacher, in the amount of 0.40% by weight of the material.

To reinforce it, a raster calender was used provided with a roller with U 3343B pattern and the applied technological characteristics were as follows:
Calender rollers temperature (smooth roller/embossed roller): 146/148° C.
Pressure of both rollers: 73 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 26.2 |
| Transversal tensile strength | EN 29073 T3 | N/50 mm | 10.6 |
| Longitudinal elongation at break | EN 29073 T3 | % | 65.5 |
| Transversal elongation at break | EN 29073 T3 | % | 96.5 |
| Bending stiffness | IST 90.3.2001 | mN | 153.4 |
| Liquid strike-through time | EDANA 150.5-02 | s | 2.03 |
| Run-off | WSP 80.9 (05) | g | 0.00 |

### Example 13

The nonwoven fabric is made from polypropylene and polyethylene filaments in the form of core/sheath in the ratio of 70% of polypropylene and 30% of polyethylene of a fineness of 1.5 den Its basis weight is 20 g/m² and its thickness is 0.25 mm, its volume mass thus being 80.00 kg/m³.

To reinforce it, a raster calender was used provided with a roller with U 3343B pattern and the applied technological characteristics were as follows:
Calender rollers temperature (smooth roller/embossed roller): 134/138° C.
Pressure of both rollers: 67 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 31.7 |
| Transversal tensile strength | EN 29073 T3 | N/50 mm | 14.1 |
| Longitudinal elongation at break | EN 29073 T3 | % | 74.7 |
| Transversal elongation at break | EN 29073 T3 | % | 128.0 |
| Bending stiffness | IST 90.3.2001 | mN | 174.8 |

### Example 14

The nonwoven fabric is made from polypropylene and polyethylene filaments in the form of core/sheath in the ratio of 70% of polypropylene and 30% of polyethylene of a fineness of 1.5 den. Its basis weight is 20 g/m² and its thickness is 0.25 mm, its volume mass thus being 80.00 kg/m³.

The nonwoven fabric is treated with hydrophilic lubrication, for example with lubricating agent Silastol PHP 90 from the company Schill&Seilacher, in the amount of 0.40% by weight of the material.

To reinforce it, a raster calender was used provided with a roller with U 3343B pattern and the applied technological characteristics were as follows:
Calender rollers temperature (smooth roller/embossed roller): 134/138° C.
Pressure of both rollers: 67 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 31.7 |
| Transversal tensile strength | EN 29073 T3 | N/50 mm | 14.1 |
| Longitudinal elongation at break | EN 29073 T3 | % | 74.7 |
| Transversal elongation at break | EN 29073 T3 | % | 128.0 |
| Bending stiffness | IST 90.3.2001 | mN | 174.8 |
| Liquid strike-through time | EDANA 150.5-02 | s | 2.21 |
| Run-off | WSP 80.9 (05) | g | 0.00 |

### Example 15

The nonwoven fabric is made from polypropylene filaments of a fineness of 0.5 den. Its basis weight is 12 g/m² and its thickness is 0.17 mm, its volume mass thus being 70.58 kg/m³.

To reinforce it, a raster calender was used provided with a roller with U 3343B pattern and the applied technological characteristics were as follows:
Calender rollers temperature (smooth roller/embossed roller): 147/155° C.
Pressure of both rollers: 69 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 36.5 |
| Transversal tensile strength | EN 29073 T3 | N/50 mm | 2.2 |
| Longitudinal elongation at break | EN 29073 T3 | % | 33.3 |
| Transversal elongation at break | EN 29073 T3 | % | 40.8 |
| Bending stiffness | IST 90.3.2001 | mN | 185.6 |

### Example 16

The nonwoven fabric is made from polypropylene filaments of a fineness of 0.5 den. Its basis weight is 12 g/m² and its thickness is 0.17 mm, its volume mass thus being 70.58 kg/m³.

The nonwoven fabric is treated with hydrophilic lubrication, for example with lubricating agent Silastol PHP 90 from the company Schill&Seilacher, in the amount of 0.40% by weight of the material.

To reinforce it, a raster calender was used provided with a roller with U 3343B pattern and the applied technological characteristics were as follows:
Calender rollers temperature (smooth roller/embossed roller): 147/155° C.
Pressure of both rollers: 69 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 36.5 |
| Transversal tensile strength | EN 29073 T3 | N/50 mm | 2.2 |
| Longitudinal elongation at break | EN 29073 T3 | % | 33.3 |
| Transversal elongation at break | EN 29073 T3 | % | 40.8 |
| Bending stiffness | IST 90.3.2001 | mN | 185.6 |
| Liquid strike-through time | EDANA 150.5-02 | s | 2.63 |
| Run-off | WSP 80.9 (05) | g | 0.00 |

### Example 17

The nonwoven fabric is made from polypropylene filaments of a fineness of 1.5 den. Its basis weight is 14 g/m² and its thickness is 0.19 mm, its volume mass thus being 73.68 kg/m³.

To reinforce it, a raster calender was used provided with a roller with U 3343B pattern and the applied technological characteristics were as follows:
Calender rollers temperature (smooth roller/embossed roller): 151/156° C.
Pressure of both rollers: 70 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 43.0 |
| Transversal tensile strength | EN 29073 T3 | N/50 mm | 20.7 |
| Longitudinal elongation at break | EN 29073 T3 | % | 67.8 |
| Transversal elongation at break | EN 29073 T3 | % | 73.9 |
| Bending stiffness | IST 90.3.2001 | mN | 210.0 |

### Example 18

The nonwoven fabric is made from polypropylene filaments of a fineness of 1.5 den. Its basis weight is 14 g/m² and its thickness is 0.19 mm, its volume mass thus being 73.68 kg/m³.

The nonwoven fabric is treated with hydrophilic lubricating agent Silastol PHP 90 from the company Schill&Seilacher, in the amount of 0.40% by weight of the material.

To reinforce it, a raster calender was used provided with a roller with U 3343B pattern and the applied technological characteristics were as follows:
Calender rollers temperature (smooth roller/embossed roller): 151/156° C.
Pressure of both rollers: 70 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 43.0 |
| Transversal tensile strength | EN 29073 T3 | N/50 mm | 20.7 |
| Longitudinal elongation at break | EN 29073 T3 | % | 67.8 |
| Transversal elongation at break | EN 29073 T3 | % | 73.9 |
| Bending stiffness | IST 90.3.2001 | mN | 210.0 |
| Liquid strike-through time | EDANA 150.5-02 | s | 2.90 |
| Run-off | WSP 80.9 (05) | g | 0.00 |

### Example 19

The nonwoven fabric is made from polypropylene filaments of a fineness of 1.5 den. Its basis weight is 17 g/m² and its thickness is 0.285 mm, its volume mass thus being 59.64 kg/m³.

To reinforce it, a raster calender was used provided with a roller with U 3343B pattern and the applied technological characteristics were as follows:
Calender rollers temperature (smooth roller/embossed roller): 153/157° C.
Pressure of both rollers: 72 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 55.0 |
| Transversal tensile strength | EN 29073 T3 | N/50 mm | 24.5 |
| Longitudinal elongation at break | EN 29073 T3 | % | 78.6 |
| Transversal elongation at break | EN 29073 T3 | % | 71.9 |
| Bending stiffness | IST 90.3.2001 | mN | 418.0 |

### Example 20

The nonwoven fabric is made from polypropylene filaments of a fineness of 1.5 den. Its basis weight is 17 g/m² and its thickness is 0.285 mm, its volume mass thus being 59.64 kg/m³.

The nonwoven fabric is treated with hydrophilic lubrication, for example with lubricating agent Silastol PHP 90 from the company Schill&Seilacher, in the amount of 0.40% by weight of the material.

To reinforce it, a raster calender was used provided with a roller with U 3343B pattern and the applied technological characteristics were as follows:
Calender rollers temperature (smooth roller/embossed roller): 153/157° C.
Pressure of both rollers: 72 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 55.0 |
| Transversal tensile strength | EN 29073 T3 | N/50 mm | 24.5 |
| Longitudinal elongation at break | EN 29073 T3 | % | 78.6 |
| Transversal elongation at break | EN 29073 T3 | % | 71.9 |
| Bending stiffness | IST 90.3.2001 | mN | 418.0 |
| Liquid strike-through time | EDANA 150.5-02 | s | 3.21 |
| Run-off | WSP 80.9 (05) | g | 0.0 |

### Example 21

The nonwoven fabric is made from polypropylene filaments of a fineness of 1.5 den. Its basis weight is 17 g/m² and its thickness is 0.28 mm, its volume mass thus being 60.71 kg/m³.

To reinforce it, a raster calender was used provided with a roller with U 3343B pattern and the applied technological characteristics were as follows:
Calender rollers temperature (smooth roller/embossed roller): 142/147° C.
Pressure of both rollers: 73 N/mm
Achieved characteristics of the nonwoven fabric:

| | measurement | | |
|---|---|---|---|
| Characteristic: | method | units | value |
| Longitudinal tensile strength | EN 29073 T3 | N/50 mm | 34.7 |
| Transversal tensile strength | EN 29073 T3 | N/50 mm | 18.0 |
| Longitudinal elongation at break | EN 29073 T3 | % | 82.3 |
| Transversal elongation at break | EN 29073 T3 | % | 83.8 |
| Bending stiffness | IST 90.3.2001 | mN | 202.2 |
| Tear strength transversal | PH 260/007 | N | 22.3 |
| Tear strength longitudinal | PH 260/007 | N | 20.9 |

where PH designates the measuring standards by the firm Paul Hartmann

This nonwoven fabric is designed to be used in the final product, for example in diapers or incontinence pads as the back-sheet. At the same time, it is used as a loop counter-fastener for the current hook-type fastening system.

### Example 22

The nonwoven fabric is made from polypropylene filaments of a fineness of 1.5 den. Its basis weight is 25 g/m² and its thickness is 0.21 mm, its volume mass thus being 119.1 kg/m³.

To reinforce it, a raster calender was used provided with a roller with U 3343B pattern and the applied technological characteristics were as follows:
Calender rollers temperature (smooth roller/embossed roller): 168/164° C.
Pressure of both rollers: 70 N/mm

At the same time as this now-woven fabric, a nonwoven fabric made of polypropylene filaments of fineness higher than 1.5 den is being produced on another production line. Its basis weight is 25 g/m² and its thickness is 0.27 mm, its volume mass thus being 92.6 kg/m³.

To reinforce it, a raster calender was used provided with a roller with the classical U 2888 pattern and the applied technological characteristics were as follows:
Calender rollers temperature (smooth roller/embossed roller): 170/174° C.
Pressure of both rollers: 70 N/mm
Achieved characteristics of the nonwoven fabric:

| | | | value | |
|---|---|---|---|---|
| | measurement | | | clas- |
| Characteristic: | method | units | bulky | sical |
| Longitudinal tensile strength | EN 29073 T3 | N/50 mm | 55.0 | 59.0 |
| Transversal tensile strength | EN 29073 T3 | N/50 mm | 27.0 | 39.0 |
| Longitudinal elongation at | EN 29073 T3 | % | 50.0 | 77.0 |
| break | | | | |
| Transversal elongation at | EN 29073 T3 | % | 65.0 | 82.0 |
| break | | | | |
| Tear strength | PH 260/007 | N | 35.0 | 19.0 |

Comparing the above results, namely the “tear strength”, it is evident that the values of the bulkier nonwoven fabric are in this characteristic higher, i.e. this nonwoven fabric can serve much better as a counter-fastener to the current hook-type fastening system than the nonwoven fabric reinforced by means of the classical pattern.

1. A nonwoven fabric of a basis weight of 10 to 30 g/m² comprising:
endless spun-melt polyolefin filaments of 0.5 to 3.5 den of fineness by wherein the fabric has a volume mass in the range of 40 to 80 kg/m³, a basis weight in the range of 10 to 30 g/m² and a thickness in the range of 0.15 to 0.80 mm, the fabric being bonded on a part of its surface in the range of 10 to 16% of its total surface area.

2. A nonwoven fabric according to claim 1, wherein it is thermal bonded on 14% of its total surface area.

3. A nonwoven fabric according to claim 1, wherein the fabric is treated with hydrophilic lubrication.

4. A nonwoven fabric according to claim 1, wherein the thermal bonding is accomplished in the form of longitudinal spots distributed in the shape of a raster, wherein each line is offset with respect to adjacent lines by a half of the longitudinal pitch of the raster, the pitch of the individual bonding spots in the longitudinal direction being equal to 2 mm and in the transversal direction being equal to 5.58 mm, the width of the bonding spot is 0.4 mm and its length is equal to 3.99 mm, the number of points per square cm is 9 points, the bonding area is 1.562 mm², the raster depth is 0.7 mm.

5. A nonwoven fabric according to claim 1, wherein the endless polyolefin filaments are monomeric.

6. A nonwoven fabric according to claim 1, wherein the endless polyolefin filaments are bicomponent filaments.

7. A method for producing the nonwoven fabric of claim 1, comprising:
creating a basic system of endless filaments of 0.5 to 3.5 den of fineness by means of a spun-melt technology;
stochastically depositing the basic system on a moving belt, its basis weight being 10 to 30 g/m²; and
passing the basis system through a calender system consisting of a smooth roller and an embossed roller.

8. The method for producing the nonwoven fabric according to claim 7, wherein a surface of the embossed roller has a temperature in the range from 110 to 190° C. and a pressure of the embossed roller on the smooth roller is in the range 50 to 100 N/mm.

9. The method for producing the nonwoven fabric according to claim 7, wherein the endless filaments are created by monopolymeric polyolefin endless filaments.

10. (canceled)

11. A method for producing the nonwoven fabric of claim 2, comprising:
creating a basic system of endless filaments of 0.5 to 3.5 den of fineness by means of a spun-melt technology;
stochastically depositing the basic system on a moving belt, its basis weight being 10 to 30 g/m²; and
passing the basis system through a calender system consisting of a smooth roller and an embossed roller.

12. A method for producing the nonwoven fabric of claim 3, comprising:
creating a basic system of endless filaments of 0.5 to 3.5 den of fineness by means of a spun-melt technology;
stochastically depositing the basic system on a moving belt, its basis weight being 10 to 30 g/m²; and
passing the basis system through a calender system consisting of a smooth roller and an embossed roller.

13. A method for producing the nonwoven fabric of claim 4, comprising:
creating a basic system of endless filaments of 0.5 to 3.5 den of fineness by means of a spun-melt technology;
stochastically depositing the basic system on a moving belt, its basis weight being 10 to 30 g/m²; and
passing the basis system through a calender system consisting of a smooth roller and an embossed roller.

14. A method for producing the nonwoven fabric of claim 5, comprising:
creating a basic system of endless filaments of 0.5 to 3.5 den of fineness by means of a spun-melt technology;
stochastically depositing the basic system on a moving belt, its basis weight being 10 to 30 g/m²; and
passing the basis system through a calender system consisting of a smooth roller and an embossed roller.

15. A method for producing the nonwoven fabric of claim 6, comprising:
creating a basic system of endless filaments of 0.5 to 3.5 den of fineness by means of a spun-melt technology;
stochastically depositing the basic system on a moving belt, its basis weight being 10 to 30 g/m²; and
passing the basis system through a calender system consisting of a smooth roller and an embossed roller.

16. A hygienic product comprising:
a first portion;
a back sheet formed from the fabric of claim 1; and
a “hook-loop” fastening system including a loop-type counter-fastener formed from the fabric of claim 1.

17. A hygienic product comprising:
a first portion;
a back sheet formed from the fabric of claim 5; and
a “hook-loop” fastening system including a loop-type counter-fastener formed from the fabric of claim 5.

18. A hygienic product comprising:
a first portion;
a back sheet formed from the fabric of claim 6; and
a “hook-loop” fastening system including a loop-type counter-fastener formed from the fabric of claim 6.

19. A hygienic product comprising:
a first portion;
a back sheet formed from the fabric of claim 7; and
a “hook-loop” fastening system including a loop-type counter-fastener formed from the fabric of claim 7.
